(19) United States
(12) Patent Application Publication
(10) Pub. No.: US 2023/0157632 A1
(43) Pub. Date: May 25, 2023

(54) Detecting Obstructive Sleep Apnea/Hypopnea Using Micromovements

(71) Applicants: Anhui Huami Health Technology Co., Ltd., Hefei (CN); Zepp, Inc., Cupertino, CA (US)

(72) Inventors: Yan Vule, Port Moody (CA); Vahid Zakeri, Vancouver (CA); Artem Galeev, Vancouver (CA); Kongqiao Wang, Hefei, Anhui FTZ (CN)

(21) Appl. No.: 17/531,047

(22) Filed: Nov. 19, 2021

(51) Int. Cl. A61B 5/00; A61B 5/113; G16H 20/40

(52) U.S. Cl. CPC A61B 5/4818; A61B 5/113; A61B 5/7267; A61B 5/7282; G16H 20/40; A61B 2562/0219

(57) ABSTRACT

Apnea-hypopnea detection includes obtaining accelerometer data from an accelerometer configured to measure micro-movements that are due to respiration. Displacement values are obtained from the accelerometer data. Features are obtained using the accelerometer data. An apnea-hypopnea index (AHI) is obtained from a machine learning model that uses the features as inputs. The displacement values correspond to peaks in the accelerometer data.

### FIELD

The present disclosure relates generally to apnea and hypopnea detection, more specifically, to using micromovements detected by an accelerometer to detect apnea and hypopnea.

### SUMMARY

A first aspect is a method for apnea-hypopnea detection. The method includes obtaining accelerometer data from an accelerometer configured to measure micro-movements that are due to respiration; obtaining displacement values from the accelerometer data; obtaining features using the accelerometer data; and obtaining an apnea-hypopnea index (AHI) from a machine learning model that uses the features as inputs. The displacement values correspond to peaks in the accelerometer data.

A second aspect is a device for apnea-hypopnea detection. The device includes a processor configured to execute instructions to obtain accelerometer data from an accelerometer configured to measure micro-movements that are due to respiration; obtain displacement values from the accelerometer data; obtain features using the accelerometer data; and obtain an apnea-hypopnea index (AHI) from a machine learning model that uses the features as inputs. The displacement values correspond to peaks in the accelerometer data.

A third aspect is a non-transitory computer readable medium that stores instructions operable to cause one or more processors to perform operations for apnea-hypopnea detection. The operations include obtaining accelerometer data from an accelerometer configured to measure micro-movements that are due to respiration; obtaining displacement values from the accelerometer data; and obtaining, from a machine learning model that uses the features as inputs, respective labels for frames of the displacement values, each label indicating an apnea event, a hypopnea event, or a no-event. The displacement values correspond to peaks in the accelerometer data; obtaining features using the accelerometer data.

### BRIEF DESCRIPTION OF THE DRAWINGS

The disclosure is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

FIG. 1 depicts a perspective view of a device that is according to the teachings herein.

FIG. 2 depicts a system 200 for using micro-movements for apnea/hypopnea detection.

FIG. 3 depicts an illustrative processor-based computing device.

FIG. 4A depicts an example of raw data collected by a three-axis accelerometer calibrated to detect micro-motion during a sleep state.

FIG. 4B provides a zoomed-in view of a portion of the example raw data of FIG. 4A.

FIG. 4C provides a zoomed-in view of a portion of the example raw data of FIG. 4A.

FIG. 5 illustrates an example of a portion of an output of a single-axis accelerometer that has been pre-processed.

FIG. 6 provides an example of respiratory signals indicative of sleep apnea events.

FIG. 7 is a flowchart of an example of a technique for apnea/hypopnea detection.

FIG. 8 illustrates an example of a histogram of displacement drop ratios.

### DETAILED DESCRIPTION

Obstructive sleep apnea/hypopnea (OSAH) is a prevalent disorder that affects sleep quality. OSAH is a condition in which the upper airway is obstructed in repeated episodes (i.e., events) during sleep. When the upper airway is totally occluded, the condition is called apnea; and when the upper airway is partially occluded, the condition is called hypopnea. OSAH causes severely fragmented sleep as a result of having to wake up enough (i.e., without regaining full consciousness) to regain muscle control in the throat and to reopen the airway. OSAH raises the heart rate and increases blood pressure, which in turn place stress on the heart. OSAH results in sleepiness, fatigue, physiological and psychological distress, and various other health complications, such as cardiovascular and cerebrovascular diseases. Successful detection and treatment of OSAH can reduce the risks of ailments induced by or related to OSAH.

Polysomnography (PSG) is the gold standard in OSAH detection. Polysomnography tests are typically performed by sleep technologists at medical facilities, such as hospitals or dedicated sleep clinics. Sensors are placed on the scalp, temples, chest, and legs of an individual using adhesives. The sensors are connected by wires to a computer. A clip may also be placed on the finger or ear to monitor the level of oxygen in the blood. As such, it is, at the least, impractical, uncomfortable, and cumbersome for individuals to monitor their own sleep quality, on a nightly basis, to detect OSAH using polysomnography machines.

As is known, the Apnea/Hypopnea Index (AHI) is a metric that measures sleep apnea severity. The AHI can be calculated as the sum of the number of apneas (i.e., pauses in breathing) plus the number of hypopneas (i.e., periods of shallow breathing) that occur, on average, each hour of sleep. To count in the index, an apnea event and a hypopnea event must have a certain duration (e.g., at least 10 seconds). Based on the AHI, the severity of OSAH can be classified as follows: the sleep is classified as “normal” (or no sleep apnea), if the AHI is less than 5 events per hour; the sleep is classified as “mild sleep apnea,” if the AHI is between 5 and 15 events per hour; the sleep is classified as “moderate sleep apnea,” if the AHI is between 15 and 30 events per hour; and the sleep is classified as “severe sleep apnea,” if the AHI is greater than 30 events per hour.

In addition to other physiological changes in the body, and according to the American Academy of Sleep Medicine (AASM), the amplitude of respiration is reduced by more than 90% and 30% compared to normal breathing for at least 10 seconds during an apnea event and a hypopnea event, respectively. The heart rate decreases with each OSAH event. During an OSHA event, a relative bradycardia (i.e., a slower than normal heart rate) is observed. After the end of the OSAH event, when respiration is restored, a relative tachycardia (i.e., a fast heart rate) is observed. Oxygen saturation in the blood drops with the cessation of respiration and is restored during the few restituting breaths.

During respiration, the lungs fill and contract therewith lifting and lowering the chest. As such, direct respiration amplitude measurements can be obtained on the chest. Respiration amplitude of a respiration signal is a measure of the wave from its height from the peak (inhalation) to the crest (exhalation).

Accordingly, respiration amplitude changes can be used to calculate the AHI. Measuring the respiration amplitude changes can be performed by attaching one or more devices that include sensors on an individual's chest and measuring the shifts between inhalations and exhalations. However, such devices may be uncomfortable and inconvenient for personal use. Additionally, it may not be possible for individuals to securely fasten such devices to their chests so that the devices are tolerant to movements (e.g., tossing and turning) during sleep. Improper or insecure placement of such devices can result in faulty and inaccurate measurements.

Respiration also causes at least micro movements in at least some parts of the body. Such micro movements can be measured using an accelerometer that may be embedded in a wearable device.

Implementations according to this disclosure use a comfortable and convenient wearable device to indirectly measure respiration amplitude changes using accelerometer data obtained from an accelerometer of the wearable device. The wearable device, which includes the accelerometer, can be secured to a sleeping individual such that the accelerometer can be tolerant to the movements or orientations during sleep. The wearable device can be a wrist watch, ear buds, a headphone, a bracelet, an ankle bracelet, and the like.

Indirectly measuring respiration amplitude changes, as further described below, includes obtaining a set of displacement values that may be descriptive of, indicative of, or correlated to, respiration amplitude changes. As such, the displacement values can be measured properties that relate to the respiration amplitude changes. As further described herein, accelerometer data can be used to obtain displacement values, such as of a body part, and which can be related to or correlated to respiration amplitudes. The displacement values can be used to obtain OSAH statuses. For example, the displacement values can be used to obtain features that can then be used to obtain labels associated with apnea/hypopnea event, an AHI index, or both. The OSAH statuses can be obtained using a machine learning (ML) model that receives the features extracted from the displacement values as input and outputs, for example, an AHI. It is noted that displacement values obtained using micro-movements detected by an accelerometer, as described herein, may not be obtainable using other types of sensors, such as electrocardiogram (ECG) sensors.

Disclosed herein are devices and techniques for sensing, measuring, analyzing, displaying physiological information, or a combination thereof. The physiological information includes OSAH events. In one aspect, a wearable device comprising at least one of an upper module or a lower module includes an accelerometer for detecting micro-movements associated with or caused by breathing. The wearable device may be worn on a body of a person (also referred to herein as a wearer or user) such that one or more sensors of the upper and lower modules contact a targeted area of tissue. In one implementation, the wearable device is a watch, band, or strap that can be worn on the wrist of a user such that the upper and lower modules are each in contact with a side of the wrist.

The techniques described herein provide a simple and user friendly solution to the problem of apnea/hypopnea detection (such as compared to Polysomnography). Also, via providing new features that are obtained using displacement values obtained from the accelerometer data, OSAH may be accurately detected or, at least, more accurately detected than other conventional techniques of detecting OSAH using wearable devices.

While the systems and devices described herein may be depicted as wrist worn devices, one skilled in the art will appreciate that the systems and methods described below can be implemented in other contexts, including the sensing, measuring, analyzing, and display of physiological data gathered from a device worn at any suitable portion of a user's body, including but not limited to, other portions of the arm, other extremities, the head, the chest, the abdomen or mid-section, or a combination thereof.

The processor functions to analyze acceleration data, velocity data, or both and to remove or isolate some of the constituents from the acceleration data, velocity data, or both. The processor may subtract, remove, isolate, or a combination thereof the first measurement from the second measurement. The processor may process data along three axes of the acceleration data, the velocity data, or both. The processor may weigh data from the acceleration data, the velocity data, or both. Respiration rates or features correlated thereto may be derived from movements (e.g., micro-movements) of a body part of a user. The features may be determined by (i.e., obtained from) movements of the device caused by breathing movements. The features may be derived by monitoring movements of a user without knowing a position of the device relative to the user, a position of the user, or both.

Reference will now be made in detail to certain illustrative implementations, examples of which are illustrated in the accompanying drawings. Wherever possible, the same reference numbers will be used throughout the drawings to refer to the same or like items.

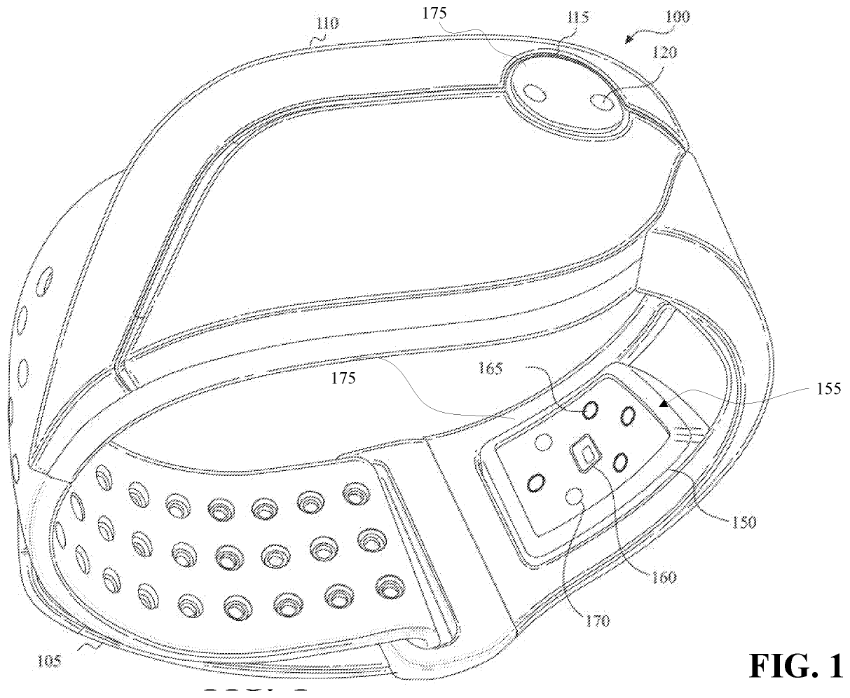

FIG. 1 depicts a perspective view of a device 100 that is according to the teachings herein. The device 100 may be a physiological monitor worn by a user to at least one of sense, collect, monitor, analyze, or display information pertaining to one or more physiological characteristics to provide physiological information. The device 100 comprises a band, strap, or wristwatch. The device 100 is a wearable monitoring device configured for positioning at a user's wrist, arm, another extremity of the user, or some other area of the user's body.

The device 100 may comprise at least one of an upper module 110 or a lower module 150, each comprising at least one of one or more sensing tools including sensors and processing tools for detecting, collecting, processing, or displaying one or more physiological parameters and/or physiological characteristics of a user and/or other information that may or may not be related to health, wellness, exercise, sleep, or physical training sessions (e.g., characteristic information).

The upper module 110 and the lower module 150 of the device 100 may comprise a strap or band 105 extending from opposite edges of each module for securing device 100 to the user. The band(s) 105 may comprise an elastomeric material or the band(s) 105 may comprise some other suitable material, including but not limited to, a fabric or metal material.

Upper module 110 or lower module 150 may also comprise a display unit (not shown) for communicating information to the user (i.e., the wearer of the device). The display unit may be an LED indicator comprising a plurality of LEDs, each a different color. The LED indicator can be configured to illuminate in different colors depending on the information being conveyed. For example, where device 100 is configured to monitor at least one of the user's heart rate or respiration rate, the display unit may illuminate light of a first color when at least one of the user's hear rate or respiration rate is in a first numerical range, illuminate light of a second color when at least one of the user's hear rate or respiration rate is in a second numerical range, and illuminate light of a third color when at least one of the user's hear rate or respiration rate is in a third numerical range. In this manner, a user may be able to detect his or her approximate heart rate and/or respiration rate at a glance, even when numerical heart rate information and/or respiration rate information is not displayed at the display unit, and/or the user only sees device 100 through the user's peripheral vision.

The display unit may comprise a display screen for displaying images, characters, graphs, waveforms, or a combination thereof to at least one of the user or a medical professional. The display unit may further comprise one or more hard or soft buttons or switches configured to accept input by the user. The display unit may switch or be toggled between displaying user physiological information.

The device 100 may further comprise one or more communication modules. Each of the upper module 110 and the lower module 150 may comprise a communication module such that information received at either module can be shared with the other module. One or more communication modules may also communicate with other devices such as personal device of the user (such as a handheld device, a smart phone, a tablet, a laptop computer, a desktop computer, or the like) or a server (such as a cloud-based server). The communications between the upper and lower modules can be transmitted from one module to the other wirelessly (e.g., via Bluetooth, RF signal, Wi-Fi, near field communications, etc.) or through one or more electrical connections embedded in band 105. Any analog information collected or analyzed by either module can be translated to digital information for reducing the size of information transfers between modules. Similarly, communications between either module and device can be transmitted wirelessly or through a wired connection, and translated from analog to digital information to reduce the size of data transmissions.

As shown in FIG. 1, lower module 150 can comprise an array of sensor array 155 including but not limited to one or more optical detectors 160, one or more light sources 165, one or more contact pressure/tonometry sensors 170, and at least one of the one or more gyroscopes or accelerometers 175. These sensors are only illustrative of the possibilities, however, and lower module may comprise additional or alternative sensors such as one or more acoustic sensors, electromagnetic sensors, ECG electrodes, bio impedance sensors, or galvanic skin response, or a combination thereof. Though not depicted in the view shown in FIG. 1, upper module 110 may also comprise one or more such sensors and components on its inside surface, i.e., the surface in contact with the user's tissue or targeted area.

The location of sensor array 155 or the location of one or more sensor components of sensor array 155 with respect to the user's tissue may be customized to account for differences in body type across a group of users or placement in different locations on a user. For example, band 105 may comprise an aperture or channel within which lower module 150 is movably retained. In one implementation, lower module 150 and channel can be configured to allow lower module 150 to slide along the length of channel using, for example, a ridge and groove interface between the two components. For example, if the user desires to place one more components of sensor array 155 at a particular location on his or her wrist, or mid-section, the lower module 150 can be slid into the desired location along band 105. Though not depicted in FIG. 1, band 105 and upper module 110 can be similarly configured to allow for flexible or customized placement of one or more sensor components of upper module 110 with respect to the user's wrist or targeted tissue area.

The sensors and components proximate or in contact with the at least one of the user's tissue, upper module 110, or lower module 150 may comprise additional sensors or components on their respective outer surfaces, i.e., the surfaces facing outward or away from the user's tissue. In the implementation depicted in FIG. 1, upper module 110 comprises one such outward facing sensor array 115. The sensor array 115 may comprise one or more ECG electrodes 120, and/or one or more gyroscopes and/or accelerometers 175. Similar to the sensor arrays of the upper and lower modules proximate or in contact with the user's tissue, outward facing sensor array 115 may further comprise one or more contact pressure/tonometry sensors, photo detectors, light sources, acoustic sensors, electromagnetic sensors, bio impedance sensors, accelerometer, gyroscope, and/or galvanic skin response sensors.

The outward facing sensors of sensor array 115 can be configured for activation when touched by the user (with his or her other hand) and used to collect additional information. The outward facing sensors may measure without being in direct contact with the user. The outward facing sensors of sensor array 115 may be an accelerometer 175 and the accelerometer 175 may indirectly monitor movements or micro-movements (e.g., an acceleration or a velocity change) that are transmitted to the sensor through the band or the module moving or being moved or a gyroscope that monitors velocities to determine micro-movements. In an example, where lower module 150 comprises one or more optical detectors 160 and light sources 165 for collecting ECG, PPG, or heart rate information of the user, outward facing sensor array 115 of upper module 110 may comprise ECG electrodes 120 that can be activated when the user places a fingertip in contact with the electrodes. While the optical detectors 160 and light sources 165 of lower module 150 can be used to continuously monitor blood flow of the user, outward facing sensor array 115 of upper module 110 can be used periodically or intermittently to collect potentially more accurate blood flow information which can be used to supplement or calibrate the measurements collected and analyzed by an inward facing sensor array, the sensor array 155, of lower module 150.

In addition to the inward and outward facing sensors, device 100 may further comprise additional internal components such at least one of the as one or more accelerometers or gyroscopic components for determining whether and to what extent the user is in motion (i.e., whether the user is walking, jogging, running, swimming, sitting, or sleeping), breathing rhythm, breathing signals, or a combination thereof of a user. Information collected by at least one of the accelerometer(s) or gyroscopic components can also be used to calculate the number of steps a user has taken over a period of time. The activity information may measure movements. The movements measured may be macro-movements such as walking or jogging. The movements may be micro-movements.

The micro-movements may be caused by a surface of a user's skin or body part being moved due to respiration, heartbeat, or a both. The micro-movements may have a displacement (e.g., length) less than a predetermined displacement in order for at least one of the accelerometer or gyroscope to at least one of the measure or record the micro-movements. For example, when a user walks the accelerometer may measure a movement of more than 1 cm, when the accelerometer detects a user heart beat the accelerometer may measure a displacement of between 4 mm and 1 cm, and when the accelerometer measures a displacement of 4 mm or less (e.g., a micro-movement). The micro-movements may be charted in wave form such that the micro-movements are charted with a peak and a valley.

The displacement values may assist a non-transitory computer readable medium or processor in isolating movements caused by multiple sources (e.g., heart beat and respiration). The processor may receive data from at least one of the accelerometer or gyroscope related to movements of the user. The processor may dynamically filter the data. The processor may provide a respiratory signal regarding the respiration of the user (referred to herein also as acceleration data). The processor may analyze the acceleration data without regard to a position of the device relative to the user or a position of the user. The processor may filter out unwanted signals and isolate only desired signals. For example, the processor may learn which signals are of interest and the processor may analyze only those signals of interest. The processor may be in communication with or include a non-transitory computer-readable medium.

At least one of the upper or lower modules 110 or 150 can be configured to continuously collect data from a user using an inward facing sensor array. However, certain techniques can be employed to reduce power consumption and conserve battery life of device 100. For instance, only one of the upper or lower modules 110 or 150 may continuously collect information. The module may be continuously active, but may wait to collect information when conditions are such that accurate readings are most likely.

For example, when one or more accelerometers or gyroscopic components of device 100 indicate that a user is still, at rest, or sleeping, one or more sensors of at least one of the upper module 110 or lower module 150 may collect information from the user while artifacts resulting from physical movement are absent. The accelerometer or gyroscope may not begin reading until the heart rate of the user measured by another sensor is below a predetermined limit. For example, if the ECG or PPG demonstrates that the user is moving then, the accelerometer or gyroscope may not be turned on. In another example, the accelerometer or gyroscope may turn off if macro-movements are detected or a number of macro-movements are detected above a threshold amount (e.g., 5 or more per min, 10 or more per min, 20 or more per min, 30 or more per min, or 60 or more per minute). The processor may be configured to remove or filter out macro-movements. Thus, the accelerometer or gyroscope may only measure micro-movements if the macro-movements are below the threshold amount (e.g., 20 or less per minute, 10 or less per minute, 5 or less per minute, or 2 or less per minute). Thus, the accelerometer or gyroscope when set, placed, or configured to read micro-movements may only be activated when macro-movements are not present or when macro-movements are infrequent. The accelerometer or gyroscope may measure micro-movements and macro-movements simultaneously and the macro-movements may be considered outliers and may be removed from reporting. Data provided by at least one of the accelerometer or gyroscope may include an x-component, a y-component, a z-component, or a combination of the x/y/z-components within a coordinate system.

The physiological information from an upper module 110, a lower module 150, or both may be graphically displayed or represented by a waveform on a display (not shown) of the device 100. The graphical display may be provided as an output. The output may include physiological information of a user. For example, the information collected may be categorized and then graphically represented as an output or two or more outputs. The one or more outputs may be one or more waveforms, two or more waveforms, or three or more waveforms. The waveforms may be individually created. The waveforms may overlay one another. The waveforms may be created by categorizing the micro-movements. The micro-movements may be categorized by strength of the micro-movements, frequency of the micro-movements, duration of the micro-movements, or a combination thereof. The waveforms may be a one or more waveforms such as a sine wave or a sinusoidal pattern. The output may have one graph having respiration signals and a graph having a heart rate.

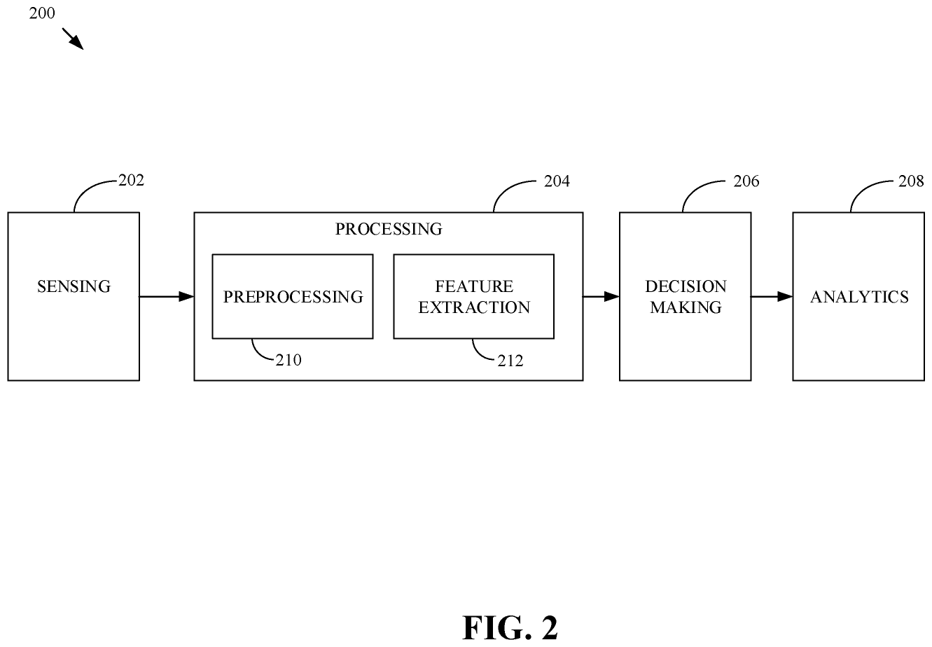

FIG. 2 depicts a system 200 for using micro-movements for apnea/hypopnea detection. As shown, the system 200 implements or includes a sensing tool 202, a processing tool 204, a decision making tool 206, and an analytics tool 208. In some implementations, some of the tools may be combined, some of the tools may be split into more tools, or a combination thereof.

The tools of the system 200 may be differently configured or included in different devices. In an example, the tools 202-208 may be implemented or included in a single device, such as a wearable device that can be the device 100 of FIG. 100. In an example, the tools 202-206 may be implemented or included in a wearable device that is in communication with another device that implements or includes the analytics tool 208. The other device can be a hand-held device, a tablet, a desktop device, a network based server (e.g., a cloud-based server), or the like. In an example, the tools 202-204 may be implemented or included in a wearable device and at least one of tools 206-208 may be implemented or included in another device. In an example, the sensing tool 202 may be implemented or included in a wearable device and the tools 204-208 may be implemented or included one or more other devices. In an example, the sensing tool 2020 may be included in a wearable device that is in communication with a personal device, which includes the processing tool 204 and the decision making tool 206, which in turn is in communication with a server, which includes the analytics tool 208. Other configurations of the tools 202-208 are possible.

Devices (e.g., one or more of a wearable device, a personal device, and a server) implementing or including the tools 202-208 can communicate via wired or a wireless connections. A wired connection can be a Universal Serial Bus (USB) connection, a firewire connection, or the like. A wireless connection can be via a network using Bluetooth communications, infrared communications, near-field communications (NFCs), a cellular data network, or an Internet Protocol (IP) network.

The sensing tool 202 can include or be a sensing unit. The sensing unit includes an accelerometer (e.g., a 3D accelerometer). The sensing unit may include other sensors, as described with respect to FIG. 1. As such, the sensing unit may include a pulse oximeter, an electrocardiogram, or other sensors. As already mentioned, the sensing tool 202 and sensing unit are included in a wearable device that is worn on the body during the sleep. In an example, the device can be a wrist watch, such as the device 100 of FIG. 1. The sensing tool 202 can be used to configure the accelerometer. For example, the sensing tool can be used to configure a sensitivity of the accelerometer, to turn on or off the accelerometer, and the like. The accelerometer may be configured by a user (such as the wearer of the wearable device) or automatically configured to collect micro-movements. In an example, in response to other tools of the wearable device detecting that the user is attempting to go to sleep (such as by detecting a body position, a breathing rate, an absence of macro-movements, or some other conditions), the accelerometer can be enabled to generate an accelerometer signal corresponding to micro-movements. Regardless of how the accelerometer is enables, the sensing tool 202 detects or obtains accelerometer signals associated with or due to micro-movements, as described herein.

The sensing tool 202 can receive signals detected by the accelerometer and transmit the accelerometer signals to the processing tool 204. In an example, the accelerometer signals may be analog signals. In another example, the accelerometer signals may be sampled prior to transmission to the processing tool 204. In an example, the accelerometer signals may be directly received by the processing tool 204. Depending on the configuration of the system 200, the accelerometer signal may be transmitted to the processing tool 204 via wired communication, wireless communication, or via some other communication mechanism known to a person skilled in the art.

The processing tool 204 is depicted as including a preprocessing tool 210 and a feature extraction tool 212. The preprocessing tool 210 may be implemented by or included in the wearable device that includes the sensing tool 202, or another device (e.g., a handheld device), or a cloud-based system. The processing tool 204 analyzes the accelerometer signal to detect changes that correspond to or correlate with OSAH.

The preprocessing tool 210 performs signal processing on the accelerometer signal. Any known signal processing techniques can be performed on (e.g., applied to) the accelerometer signal to obtain accelerometer data. For example, the preprocessing tool 210 can normalize, scale, or both the accelerometer signal to reduce the effect of noise and artifacts. The preprocessing tool 210 can perform zero or more of filtering, standardization, thresholding, or other signal processing on the accelerometer signal.

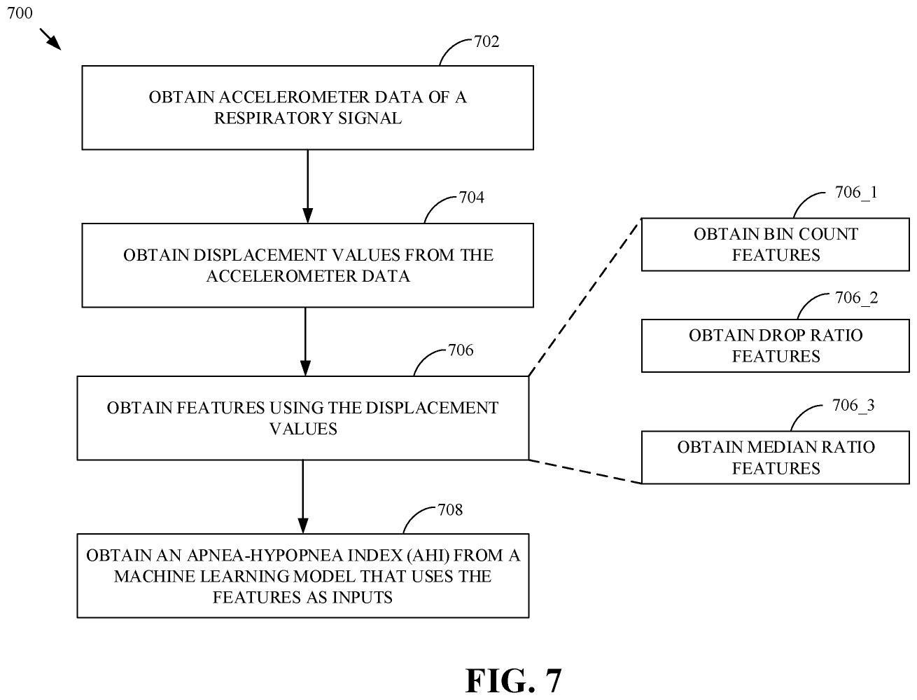

As further described with respect to FIG. 7, the processing tool 204 extracts, from the accelerometer data, features that can be used by the decision making tool 206 to obtain an AHI. Different types of features can be extracted from the accelerometer data.

The decision making tool 206 receives the features from the feature extraction tool 212 and outputs (e.g., determines, calculates, infers) an apnea/hypopnea status. In an example, the decision making tool 206 can output respective labels for windows of the accelerometer data. In an example, the labels can indicate one of the statuses “apnea event,” “hypopnea event,” or “no event.” In another example, the decision making tool 206 can output an AHI. In an example, the decision making tool 206 can output one or more labels and the AHI.

The decision making tool 206 can be or use a machine learning (ML) model that is trained to use the features as inputs and output a label, an AHI, or both. The ML model can be trained using supervised or unsupervised learning. In an example, and in the case of supervised learning, labels or AHIs for the training data may be obtained using, for example, PSG. More generally, in the case of supervised learning, the labelled training data can be previously provided by experts or certified tools (e.g. automatic algorithms in the polysomnography equipment). In the case of unsupervised learning, the ML model may be trained to recognized different distributions, which may then be interpreted, such as by a human to be specific labels or AHI values.

The ML model can be or employ one or more classifiers such as one or more of a support vector machine (SVM), a neural network, a decision tree, logistic regression, AdaBoost, XGBoost, other boosting techniques, or any other ML model that can be trained to use features, as described herein, as inputs and output a OSAH label, an AHI, or both.

The analytics tool 208 can be used to store and analyze historical accelerometer data, the corresponding outputs of the decision making tool 206, or both to provide historical insights, suggestions/recommendations, etc. regarding the OSAH statuses deduced from the historical data.

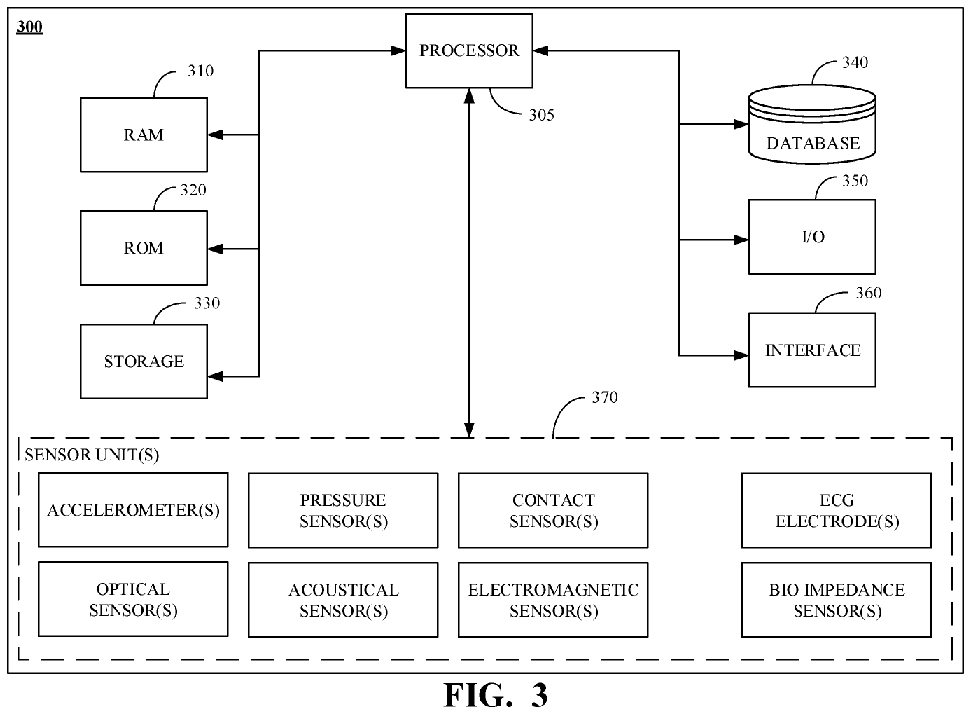

FIG. 3 depicts an illustrative processor-based, computing device 300. The computing device 300 is representative of the type of computing device that may be present in or used in conjunction with at least some aspects of device 100 or devices implementing the tools of FIG. 2, or any other device comprising electronic circuitry. For example, the computing device 300 may be used in conjunction with any one or more of transmitting signals to and from the one or more accelerometers, sensing or detecting signals received by one or more sensors of device 100, processing received signals from one or more components or modules of device 100 or a secondary device, and storing, transmitting, or displaying information. The computing device 300 is illustrative only and does not exclude the possibility of another processor- or controller-based system being used in or with any of the aforementioned aspects of device 100. At least some aspects of the computing device 300 may be included, but others may not be or may not be used to implement tools described with respect to FIG. 2, in a device that works in conjunction with the device 100 of FIG. 1 to implement the system 200 of FIG. 2. For example, a user device or a server may or may not include one or more sensor modules 370.

In one aspect, the computing device 300 may include one or more hardware and/or software components configured to execute software programs, such as software for obtaining, storing, processing, and analyzing signals, data, or both. For example, the computing device 300 may include one or more hardware components such as, for example, a processor 305, a random-access memory (RAM) 310, a read-only memory (ROM) 320, a storage 330, a database 340, one or more input/output (I/O) modules 350, an interface 360, and the one or more sensor modules 370. Alternatively and/or additionally, the computing device 300 may include one or more software components such as, for example, a computer-readable medium including computer-executable instructions for performing techniques or implement functions of tools consistent with certain disclosed embodiments. It is contemplated that one or more of the hardware components listed above may be implemented using software. For example, the storage 330 may include a software partition associated with one or more other hardware components of the computing device 300. The computing device 300 may include additional, fewer, and/or different components than those listed above. It is understood that the components listed above are illustrative only and not intended to be limiting or exclude suitable alternatives or additional components.

The processor 305 may include one or more processors, each configured to execute instructions and process data to perform one or more functions associated with the computing device 300. The term “processor,” as generally used herein, refers to any logic processing unit, such as one or more central processing units (CPUs), digital signal processors (DSPs), application specific integrated circuits (ASICs), field programmable gate arrays (FPGAs), and similar devices. As illustrated in FIG. 3, the processor 305 may be communicatively coupled to the RAM 310, the ROM 320, the storage 330, the database 340, the I/O module 350, the interface 360, and the one or more sensor modules 370. The processor 305 may be configured to execute sequences of computer program instructions to perform various processes, which will be described in detail below. The computer program instructions may be loaded into the RAM 310 for execution by the processor 305.

The RAM 310 and the ROM 32 may each include one or more devices for storing information associated with an operation of the computing device 300 and/or the processor 305. For example, the ROM 320 may include a memory device configured to access and store information associated with the computing device 300, including information for identifying, initializing, and monitoring the operation of one or more components and subsystems of the computing device 300. The RAM 310 may include a memory device for storing data associated with one or more operations of the processor 305. For example, the ROM 320 may load instructions into the RAM 310 for execution by the processor 305.

The storage 330 may include any type of storage device configured to store information that the processor 305 may use to perform processes consistent with the disclosed embodiments.

The database 340 may include one or more software and/or hardware components that cooperate to store, organize, sort, filter, and/or arrange data used by the computing device 300 and/or the processor 305. For example, the database 340 may include user profile information, historical activity and user-specific information, physiological parameter information, predetermined menu/display options, and other user preferences. Alternatively, the database 340 may store additional and/or different information. The database 340 can be used to store accelerometer data, features extracted therefrom, outputs of the decision making tool 206 of FIG. 2, other data used or generated by the system 200 of FIG. 2, or a combination thereof.

The I/O module 350 may include one or more components configured to communicate information with a user associated with the computing device 300. For example, the I/O module 350 may comprise one or more buttons, switches, or touchscreens to allow a user to input parameters associated with the computing device 300. The I/O module 350 may also include a display including a graphical user interface (GUI) and/or one or more light sources for outputting information to the user. The I/O module 350 may also include one or more communication channels for connecting the computing device 300 to one or more secondary or peripheral devices such as, for example, a desktop computer, a laptop, a tablet, a smart phone, a flash drive, or a printer, to allow a user to input data to or output data from the computing device 300.

The Interface 360 may include one or more components configured to transmit and receive data via a communication network, such as the Internet, a local area network, a workstation peer-to-peer network, a direct link network, a wireless network, or any other suitable communication channel. For example, the interface 360 may include one or more modulators, demodulators, multiplexers, demultiplexers, network communication devices, wireless devices, antennas, modems, and any other type of device configured to enable data communication via a communication network.

The computing device 300 may further comprise the one or more sensor modules 370. In one embodiment, the one or more sensor modules 370 may comprise one or more of an accelerometer module, an optical sensor module, and/or an ambient light sensor module. Of course, these sensors are only illustrative of a few possibilities and the one or more sensor modules 370 may comprise alternative or additional sensor modules suitable for use in the device 100. It should be noted that although one or more sensor modules are described collectively as the one or more sensor modules 370, any one or more sensors or sensor modules within device 100 may operate independently of any one or more other sensors or sensor modules. Moreover, in addition to collecting, transmitting, and receiving signals or information to and from the one or more sensor modules 370 at the processor 305, any the one or more sensors of the one or more sensor module 370 may be configured to collect, transmit, or receive signals or information to and from other components or modules of the computing device 300, including but not limited to the database 340, the I/O module 350, or the interface 360.

As described above with respect to FIG. 1, the one or more accelerometers of the device 100 can be used to detect large-scale motions of a subject indicative of physical activity (e.g., steps, running, walking, swimming, etc.). The same accelerometers can be used to determine the onset of a sleep period through the detection of a lack of motion. However, the sensitivity of the accelerometer(s) that detect large-scale motions aren't sensitive enough to detect movement at the wrist (or other suitable location of the body) due to breathing. In one embodiment, upon determining that the subject is engaged in sleep, the sensitivity of the accelerometer(s) can be reconfigured to detect significantly smaller motions (“micro-motions”). Alternatively, the device 100 may comprise one or more accelerometers that are dedicated to, and configured for, detecting micro-motions while one or more other accelerometers are used to detect large-scale motions.

To detect micro-motions, an accelerometer can be configured to increase its sensitivity and sampling rate. The sensitivity of an accelerometer is expressed in terms of millivolts per G-force (mV/g). Where an accelerometer configured for large-scale motions may use 7-12 g as the denominator, an accelerometer configured for micro-motion detection may use 0.001-5.0 g. In some embodiments, an accelerometer for micro-motion detection may use 1-4 g.

Additionally, it may be advantageous to increase the sampling rate of an accelerometer for measuring micro-motions as compared to when measuring large-scale motions. For example, where a frequency of 1 Hz to 3 Hz may be sufficient to sample large-scale motions, a frequency of 5 Hz to 1 KHz may be desirable when detecting micro-motions. In some embodiments, a frequency of 5 Hz to 100 Hz may be desirable. Again, regardless of the disparate sensitivity and/or sampling frequency between accelerometer settings for measuring large-scale and micro-motions, the same accelerometer(s) in the device 100 of FIG. 1 can either be reconfigured upon detection of a sleep state, or alterative accelerometer(s) having a higher sensitivity can be activated during the sleep state. If an accelerometer that is calibrated for large-scale motions is used to measure micro-motions, the amplitude of the output signal will not be great enough for accurate analysis. Conversely, if an accelerometer calibrated for micro-motions is used to measure large-scale motions, the amplitude of the output signal will always be very large, resulting in a saturated signal that provides little useful information.

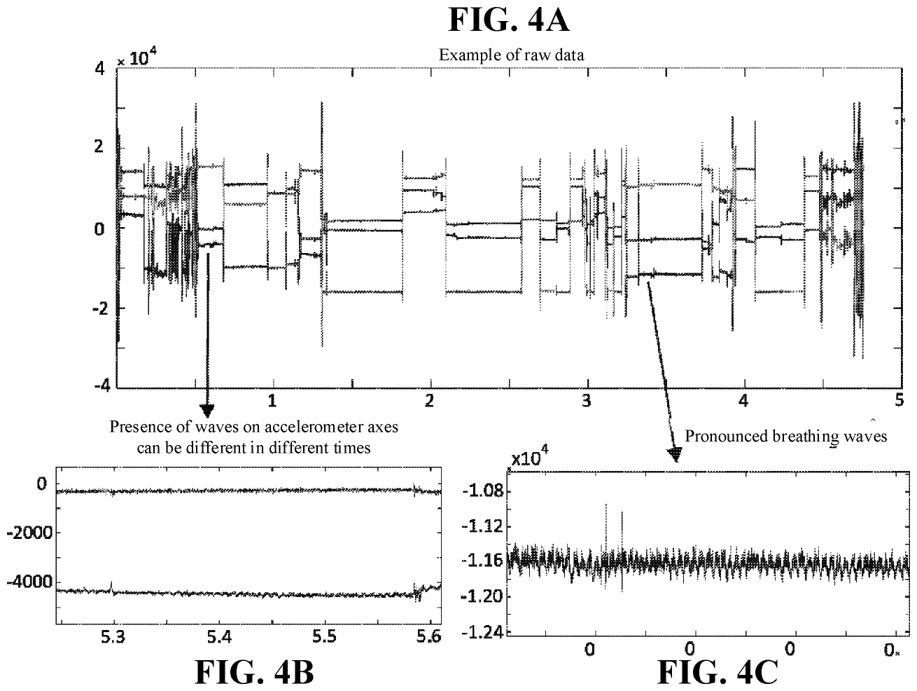

FIG. 4A depicts an example of raw data collected by a three-axis accelerometer calibrated to detect micro-motion during a sleep state. In this embodiment, the device 100 comprising the accelerometer may be located in a wearable band worn at the wrist of a user. Based on the accelerometer signals, it can be discerned when large-scale movements (such as the user shifting his/her weight, rolling over, or moving an arm) have taken place by the spikes in the accelerometer signal. Such spikes can mask the micro-motions caused by respiration. However, where the accelerometer signals are stable, the signal can be magnified, smoothed, or the like to discern a respiratory signal (i.e., the micro-movements). Moreover, where a three-axis (or two-axis) accelerometer is used, one axis may provide a more stable output signal than other axes. This disparity in the outputs of two axis of three-axis accelerometer is depicted in FIG. 4B. FIG. 4B provides a zoomed-in view of a portion of the example raw data of FIG. 4A.

At any given time during the sleep state, the output of each axis of the accelerometer can be assessed and the clearest signal (relatively higher amplitudes, relatively stable frequencies, etc.) can be selected for respiratory analysis (e.g., analysis of the displacements obtained from the accelerometer signal to obtain features as discussed below). A magnified view of a signal output from one of the accelerometer axis is depicted in FIG. 4C.

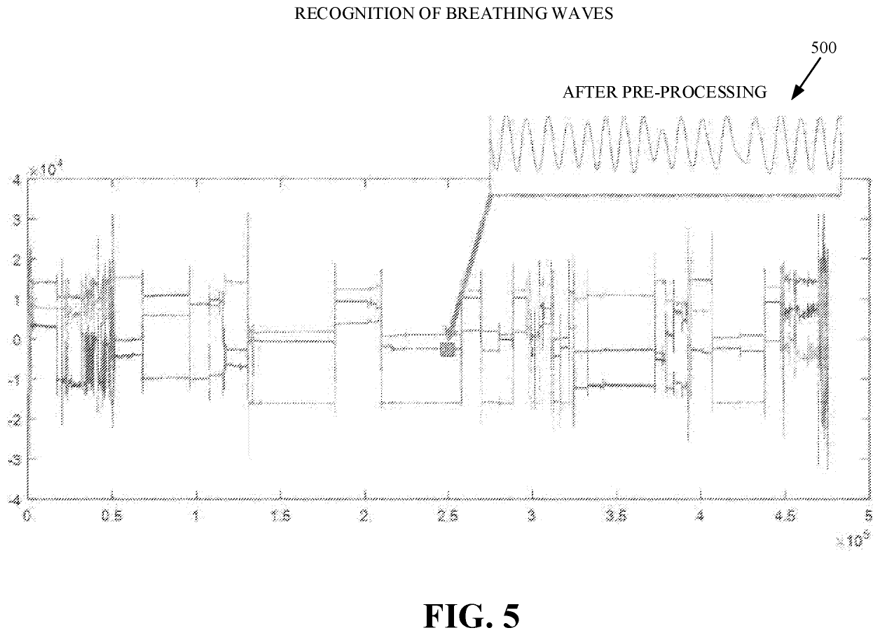

FIG. 5 illustrates an example of a portion 500 of an output of a single-axis accelerometer that has been pre-processed. The portion 500 may be obtained by the preprocessing tool 210 of FIG. 2. Thus, the corresponding accelerometer signal may be a smoothed using a smoothing filter (several of which are known) and de-noised using a de-noising filter (several methods of which are known) to obtain the portion 500 (i.e., the accelerometer data). The smoothing and/or de-noising filters, and any other processing of the accelerometer signal can be implemented using either hardware, software components, or a combination thereof.

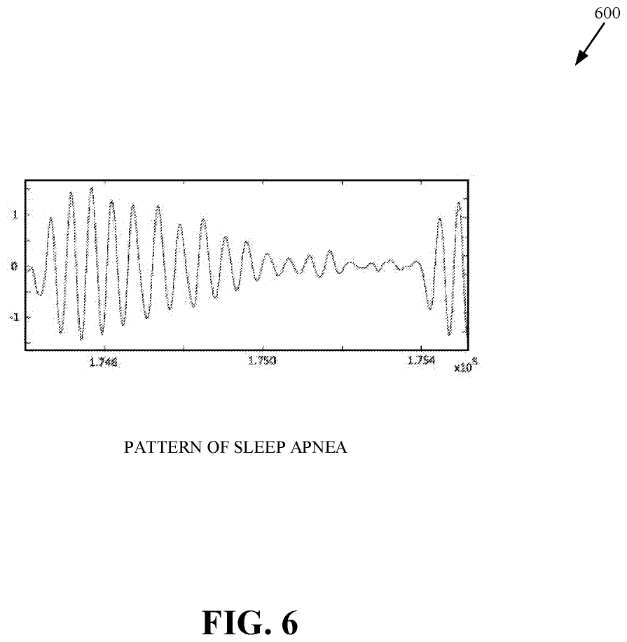

A wearable device, such as the device 100 of FIG. 1, can be configured to detect sleep events, such as OSAH events. FIG. 6 provides an example 600 of respiratory signals (i.e., accelerometer signals corresponding to micro-movement) indicative of sleep apnea events. In this situation, regular breathing tapers off and becomes very shallow until the subject needs oxygen and starts breathing normally again.

Neither of these patterns, in isolation, is indicative of sleep apnea or any other disorder. However, if numerous instances of such patterns are exhibited during a sleep state, it can be diagnosed as sleep apnea. For example, one or two instances where the accelerometer(s) of the device 100 of FIG. 1 or the sensing tool 202 of FIG. 2 output signals similar to those shown in FIG. 6 over the course of several hours would not necessarily lead to a sleep apnea diagnosis. But if the same signal patterns are experienced a number of times in a given time period (e.g. 10 times in an hour) over some threshold, or if one of the signal patterns is seen once every time period (e.g., once an hour, once every half-hour) over some percentage of the overall sleep state (e.g., 5% or 10%), then a sleep apnea diagnosis can be made.

Similarly, the device 100 of FIG. 1 or the system 200 of FIG. 2 can monitor for instances of respiratory arrest (i.e., the cessation of breathing). Respiratory arrest can be a sign of a significant problem or emergency. In one embodiment, the accelerometer(s) of the device 100 can determine that no respiration signals (i.e., no micro-movements) are detected at the user.

FIG. 7 is a flowchart of an example of a technique 700 for apnea-hypopnea detection. The technique 700 can be implemented at least in part by a device, such as the device 100 of FIG. 1. In an example, different aspects of the technique 700 can be implemented in part by respective tools of the system 200 of FIG. 2. The technique 700 can be implemented, for example, as a software program that may be executed by computing devices such as a device that may be in communication with a wearable device or receive accelerometer signals obtained using an accelerometer of the wearable device. The software program can include machine-readable instructions that may be stored in a memory such as the RAM 310, the ROM 320, or the storage 330 of FIG. 3, and that, when executed by a processor, such as the processor 305 of FIG. 3, may cause the computing device to perform the technique 700. The technique 700 can be implemented using specialized hardware or firmware.

At 702, accelerometer data of a respiratory signal can be obtained. The accelerometer data can be obtained from an accelerometer of a wearable device, such as the device 100 of FIG. 1. For example, data can be obtained from an accelerometer that is configured to detect micro-movements of a body part of a user (i.e., a wearer of the wearable device) or the micro-movements of the wearable device itself where the micro-movements are caused by breathing movements.

An accelerometer signal may be obtained from the accelerometer. Any known signal processing techniques can be performed on (e.g., applied to) the accelerometer signal to obtain the accelerometer data. For example, the preprocessing tool 210 of FIG. 3, can normalize, scale, or both the accelerometer signal to reduce the effect of noise and artifacts. The preprocessing tool 210 can perform zero or more of filtering, standardization, thresholding, or other signal processing on the accelerometer signal. The preprocessing tool 210 can select the highest signal quality from amongst the signals corresponding to the axes of the accelerometer (e.g., X, Y, and Z axes) for feature extraction. Different known techniques can be used for choosing the high quality axis, such as signal to noise ratio (SNR), signal power, zero crossing rate (ZCR), or other techniques.

At 704, displacement values are obtained from the accelerometer data. The displacement values correspond to peak values obtained from the accelerometer data. The displacement values may not have a particular unit of measure or may be said to be associated with an arbitrary unit of measure. As can be appreciated from the disclosure herein, the relation of displacement values to each other is used for feature extraction. The displacement values (i.e., the peaks) are filtered to satisfy criteria regarding the predefined maximum and minimum breathing rates. To illustrate, assume that a first peak is identified in the accelerometer data at a time t and that a second peak is identified at a time t+20 seconds. As such, the accelerometer data indicate a breathing cycle of 20 seconds, which is not possible as the normal respiration rate at rest is between 12 to 20 breaths per minute. Thus, the peak identified at the time t+20 can be discarded and not included in the displacement values.

The preprocessed accelerometer signal can be framed by a predefined time window (e.g. a rectangular window with the length of 2 minutes). Displacement values can be obtained for each of the predefined time windows. The displacement values are indicative of respiration amplitude values. In some implementations, if the displacement values within a frame are not consistent with expected breathing rates, then the whole frame is discarded (i.e., not used for feature extraction).

As mentioned above, as no direct measurements of the respiration amplitude values are available, the displacement values can be used to obtain features that may be correlated to respirations amplitude values. In an apnea event, breathing stops. When breathing stops, the heart rate also tends to gradually drop the longer the body is deprived of oxygen (i.e., the longer the apnea event). Involuntary reflexes then cause the person to startle awake and return to breathing. When breathing returns, the heart rate tends to accelerate quickly and the blood pressure tends to rise.

The displacement values are obtained from the accelerometer signal in a measurement period. The measurement period can be a time of detection of the accelerometer signal or a time of activation (e.g., manual activation) of the accelerometer to start collecting the accelerometer signal until the end of the signal (e.g., detecting an end of the signal or a deactivation of the accelerometer). The end of the signal can correspond to the person waking up. The sleep period can correspond to a period from the time that the user is detected to be asleep until the user is detected to be awake. The sleep period can correspond to one-night's sleep.

At 706, features are obtained using the displacement values. The features can be obtained using a feature extraction tool, such as the feature extraction tool 212 of FIG. 2. The obtained features are features that are pertinent (e.g., correlated) to OSAH. In an example, the features can be bin count features, as described with respect to 706_1. In an example, the features can be drop ratio features, as described with respect to 706_2. In an example, the features can be median ratio features, as described with respect to 706_3. In an example, the features can be any combination of the bin count features, the drop ratio features, or the median ratio features.

The features obtained at 706_1 are now described. A bin corresponds to, or can be thought of as, a respective consecutive number of value drops in the displacement values. The respective bin data for a bin can be obtained by associating, with the bin, a count of the respective consecutive number of value drops in the displacement values. Thus, in an example, the respective bin data can be respective counts, as described below. Said another way, a bin can be used to count a number of occurrences that meet the definition or criteria of the bin. The ordered set of displacement values constituting the consecutive decreasing values is referred herein as “drop range.”

An n-bin refers to an n-consecutive drop in displacement values, where n is an integer value in the interval [min_drops, max_drops], where min_drops is the minimum number of drops that are counted, and max_drops is the maximum number of drops that are counted; and a (max_drops+1)Plus-bin refers to a bin where a number of more than max_drops of consecutive drops in displacement values are accumulated. In an example, the minimum number of drops (min_drops) is equal to 3, and the maximum number of drops (max_drops) is equal to 6. In an example, a 3-bin, a 4-bin, a 5-bin, a 6-bin, and a 7Plus-bin (i.e., 7 or more, more than 6) may be used. However, other bins are possible. The 3-bin is used to count the number of 3 consecutive drops in displacement values. The 4-bin is used to count the number of 4 consecutive drops in displacement values. The 5-bin is used to count the number of 5 consecutive drops in displacement values. The 6-bin is used to count the number of 6 consecutive drops in displacement values. The 7Plus-bin is used to count the number of 7 or more consecutive drops in displacement values.

To illustrate, the displacement values obtained at 704 in a frame may be the values [10, 8, 7, 6, 8, 7, 8, 5, 4, 8]. The displacement values include one 4-bin consecutive drop corresponding to the consecutive values [10, 8, 7, 6]; and one 3-bin consecutive drop corresponding to the consecutive values [8, 5, 4]. As such, for this frame, 7Plus-bin=0, 6-bin=0, 5-bin=0, 4-bin=1, and 3-bin=1. To illustrate, assume that the displacement values obtained at 704 in a next frame are the displacement values [8, 8, 6, 5, 8, 5, 4, 5, 10, 9]. The displacement values include two 3-bin consecutive drops corresponding to the decreasing consecutive values [8, 6, 5] and [8, 5, 4]. Thus, for this frame, 7Plus-bin=0, 6-bin=0, 5-bin=0, 4-bin=0, and 3-bin=2. Assume further that the cumulative values of bins from the previous frames are 7Plus-bin=1, 6-bin=2, 5-bin=2, 4-bin=8, and 3-bin=10. Thus, after these 2 frames, the cumulative values become 7Plus-bin=1, 6-bin=2, 5-bin=2, 4-bin=(8+1), and 3-bin=(10+1+2). The cumulative counts can be represented as, or thought of as, the vector [1, 2, 2, 9, 13]. The cumulative counts are obtained for the measurement duration.

The bin count features can be obtained from the respective bin data by linearly scaling the respective bin to each other to be in [0, 1] interval. That is, the respective bin data can be normalized. Thus, assuming that the vector [1, 2, 2, 9, 13] contains the cumulative counts for the measurement duration, the vectors can be linearly scaled to [1/27, 2/27, 2/27, 9/27, 13/27], which constitutes the features (i.e., the bin count features). That is, the distribution of counts of consecutive drops can be obtained for a measurement period. The distribution correlates with the AHI. As such, obtaining the features at 706 can include associating with each bin a respective count of the respective consecutive number of value drops in the displacement values. As described, the counts are obtained in windows (e.g., frames) of displacement values.

In an implementation, consecutive equal displacement values cause the drop count to reset. To illustrate, given the displacement values [8, 7, 6, 6, 5, 4, 9, 8], which includes the consecutive equal displacement values [6, 6], the displacement values would include two 3 -bin drops; namely [8, 7, 6] and [6, 5, 4]. In an implementation, consecutive equal displacement values do not cause the drop count to reset. To illustrate, given the same displacement values [8, 7, 6, 6, 5, 4], the displacement values would include one 5-bin drop corresponding to the sequence [8, 7, 6, 6, 5, 4].

The drop ratio features obtained at 706_2 are now described. The drop ratio features (i.e., drop ratio values) can be obtained using displacement drop ratios. The same bins as those described with respect to the first example can be used. Thus, bins corresponding to 3, 4, 5, 6, and 7 and more value drops can be used. A displacement drop ratio value of a drop range can be computed, using equation (1), as a displacement drop divided by the number of consecutive drops in the drop range, where the displacement drop is the difference between the highest and the lowest values of a drop range divided by the highest value of the range, and where the drop range is given by [highest, . . . , lowest] and includes n elements.

$$drop\_ratio = \frac{\frac{highest - lowest}{highest}}{n} \qquad ( 1 )$$

To illustrate, given the displacement values [10, 8, 7, 6, 8, 7, 8, 5, 4, 8] in a window, which includes one 4-bin drop range [10, 8, 7, 6] and one 3-bin drop range [8, 5, 4], the displacement drop ratio value of the drop range [10, 8, 7, 6] is (10-6)/10/4, and the displacement drop ratio value of the drop range [8, 5, 4] is (8-4)/8/3.

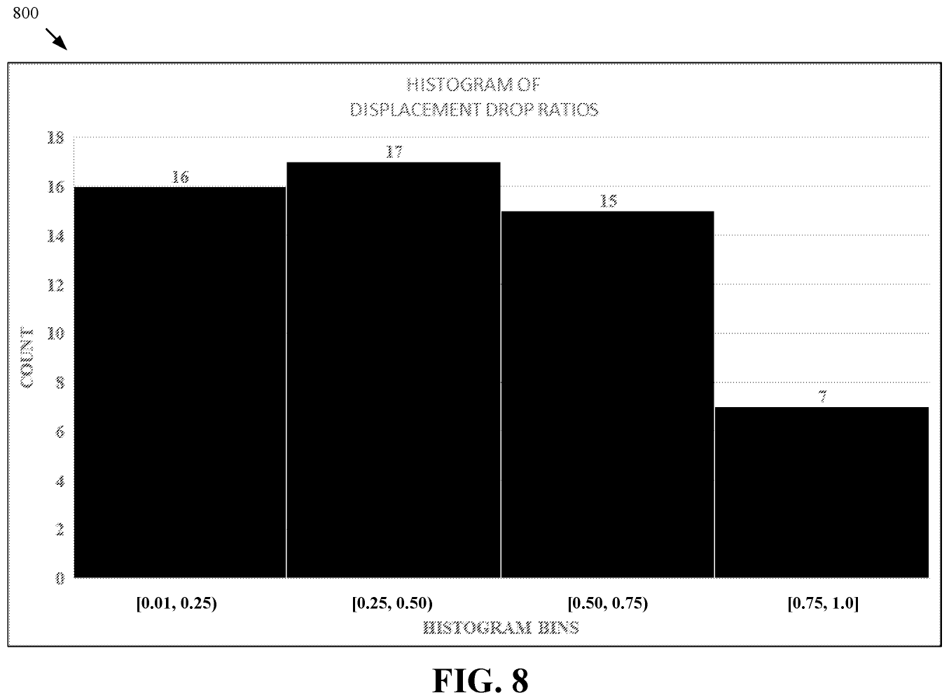

The set of displacement drop ratio values can be accumulated for the measurement duration. At the end of the measurement period, a histogram of displacement drop ratio values is obtained from the set of displacement drop ratio values for a prespecified number of histogram bins over the range [0, 1]. In an example, the histogram bins can be linearly spaced. That is, all histogram bins can have the same width. In an example, the prespecified number of histogram bins can be 10. However, other prespecified number of histogram bins are possible. The number of displacement drop ratio values in the histogram bins constitute the drop ratio features. FIG. 8 illustrates an example of a histogram 800 of displacement drop ratio values. The histogram 800 illustrates four linearly spaced histogram bins corresponding to the histogram bins [0.01, 0.25), [0.25, 0.50), [0.5, 0.75), and [0.75, 1] and that include, respectively 16, 17, 15, and 7 displacement drop ratio values. The drop ratio features can be the set of number of displacement drop ratios ordered by histogram bins; namely (16, 17, 15, 7).

As such, obtaining the features can include obtaining displacement drop ratio values using the displacement values, where a displacement drop ratio value of a drop range [highest, . . . , lowest] identified in the displacement values and including n displacement values is obtained using the formula ((highest-lowest)/highest/n). The displacement drop ratio values are obtained by processing the displacement values in frames (e.g., windows). The displacement drop ratio values are obtained for all frames of the accelerometer data obtained in the measurement period. The displacement drop ratio values are partitioned into groups. Each group includes a respective range of the displacement drop ratio values. The groups can be as described with respect to the histogram bins. The counts (numbers, cardinality) of the displacement drop ratio values in the groups can be used as the features.

The median ratio features obtained at 706_3 are now described. The accelerometer signal can be framed by a predefined window of a predefined length. For example, the window can be a rectangular window and the predefined length can be 130 seconds. Each frame is split into two subframes, a first subframe having a first duration and a second subframe having a second duration corresponding to the remaining duration of the frame. In an example, the first duration can be 120 seconds and the second duration can be 10 seconds (i.e., the predefined duration minus the first duration). The second duration can relate or be equal to a duration of an apnea event or a hypopnea event must have a certain duration that counts in the AHI. Such duration is described above as being 10 seconds. The rationale for splitting a frame into two subframes derives from the fact that the AHI is essentially a comparison of normal breathing (e.g., normal breathing rates) to breathing during an OSAH event. Thus, in obtaining the median ratio features, the first subframe is assumed to correspond to normal breathing and the second subframe is used to determine whether an OSAH event occurred during the second subframe.

The displacement values for each subframe is obtained. The ratio (i.e., a “median ratio value”) between the median of displacement values in the second subframe and the first subframe is computed. To illustrate, assume that the displacement values in a frame are [10 9 8 10 9 8 10 9 8 10 9 8 7 5 1 2 3]. The displacement values of the first subframe (e.g., the first 120 seconds) may be [10 9 8 10 9 8 10 9 8 10 9 8 7 5] and the displacement values of the second subframe (e.g., the next 10 seconds) may be [1 2 3]. The median displacement value of the first subframe is 9 and the median displacement value of the second subframe is 2. Thus, the median ratio value for this frame is 2/9.

A next frame is obtained from the accelerometer data using a rolling window using an increment. In an example, the increment may be 10 seconds. However, other increments are possible. To illustrate, and assuming that an increment of 10 seconds corresponds to 3 displacement values (that is, 3 displacement values may be obtained in 10 seconds), the next frame may be [10 9 8 10 9 8 10 9 8 7 5 1 2 3 5 6 9] where the first 3 values (i.e., 10, 9, and 8) are moved left out of the frame and 3 new values (i.e., 5, 6, and 9) are added to the tail of the frame. As such, the new first subframe is [10 9 8 10 9 8 10 9 8 7 5 1 2 3], which has a median of 8, and the new second subframe is [5 6 9], which has a median of 6. Thus, the median ratio value is 6/8.

These median ratio values are augmented for each frame in the accelerometer signal until the end of the signal. That is, the median ratio values of the frames of the measurement period are obtained. Median ratio values that are greater than 1 are discarded. A histogram of the median ratio values is obtained for a prespecified number of histogram bins. The histogram bins can be as described with respect to the drop ratio features obtained at 706_2. The number of median ratio values in the histogram bins constitute the median ratio features.

As such, obtaining the features at 706 can include partitioning the displacement values into frames using a sliding window. The displacement values are obtained from the accelerometer data in a measurement period. Median ratio values are obtained from the frames. Obtaining a median ratio value of a frame can include partitioning the frame into a first subframe that includes first displacement values and a second subframe that includes second displacement values; and obtaining the median ratio value as a ratio of a median value of the second displacement values divided by a median value of the first displacement values. The median ratio values can be partitioned into groups where each group includes a respective range of the median ratio values. The groups can be as described with respect to the histogram bins. Respective counts of the median ratio values in the groups can be used as the features.

At 708, an apnea-hypopnea index (AHI) can be obtained from a machine learning model that uses the features as inputs. The machine learning model can be as described with respect to the decision making tool 206 of FIG. 2. In an example, respective labels for the frames of the displacement values can additionally or alternatively be obtained. Each label can indicate whether an apnea event, a hypopnea event, or a no-event was inferred/detected for the frame. A count of the number of frames labeled to apnea events and hypopnea events can be used to calculate the AHI.

It may be appreciated that various changes can be made therein without departing from the spirit and scope of the disclosure. Moreover, the various features of the implementations described herein are not mutually exclusive. Rather any feature of any implementation described herein may be incorporated into any other suitable implementation.

Additional features may also be incorporated into the described systems and methods to improve their functionality. For example, those skilled in the art will recognize that the disclosure can be practiced with a variety of physiological monitoring devices, including but not limited to heart rate and blood pressure monitors, and that various sensor components may be employed. The devices may or may not comprise one or more features to ensure they are water resistant or waterproof. Some implementations of the devices may hermetically sealed.

Other implementations of the aforementioned systems and methods will be apparent to those skilled in the art from consideration of the specification and practice of this disclosure. It is intended that the specification and the aforementioned examples and implementations be considered as illustrative only, with the true scope and spirit of the disclosure being indicated by the following claims.

While the disclosure has been described in connection with certain implementations, it is to be understood that the disclosure is not to be limited to the disclosed implementations but, on the contrary, is intended to cover various modifications and equivalent arrangements included within the scope of the appended claims, which scope is to be accorded the broadest interpretation so as to encompass all such modifications and equivalent structures as is permitted under the law.

What is claimed is:

1. A method for apnea-hypopnea detection, comprising:
obtaining accelerometer data from an accelerometer configured to measure micro-movements that are due to respiration;
obtaining displacement values from the accelerometer data, wherein the displacement values correspond to peaks in the accelerometer data;
obtaining features using the accelerometer data; and
obtaining an apnea-hypopnea index (AHI) from a machine learning model that uses the features as inputs.

2. The method of claim 1, wherein obtaining the displacement values from the accelerometer data comprises:
obtaining respective displacement values in frames of the accelerometer data, wherein each frame corresponds to a predefined time window.

3. The method of claim 1, wherein obtaining the features comprises:
obtaining respective bin count data for bins,
wherein each bin corresponds to a respective consecutive number of value drops in the displacement values, and
wherein the respective bin count data for a bin is obtained by:
associating with the bin a count of the respective consecutive number of value drops in the displacement values.

4. The method of claim 1, wherein obtaining the features comprises:
obtaining displacement drop ratio values using the displacement values, wherein a displacement drop ratio value of a drop range [highest, . . . , lowest] identified in the displacement values and including n displacement values is obtained using a formula ((highest−lowest)/highest/n);
partitioning the displacement drop ratio values into groups, wherein each group includes a respective range of the displacement drop ratio values; and
using respective counts of the displacement drop ratio values in the groups as the features.

5. The method of claim 1, wherein obtaining the features comprises:
partitioning the displacement values into frames using a sliding window;
obtaining median ratio values from the frames, wherein obtaining a median ratio value of a frame comprises:
partitioning the frame into a first subframe that includes first displacement values and a second subframe that includes second displacement values; and
obtaining the median ratio value as a ratio of a median value of the second displacement values divided by a median value of the first displacement values;
partitioning the median ratio values into groups, wherein each group includes a respective range of the median ratio values; and
using respective counts of the median ratio values in the groups as the features.

6. The method of claim 5, further comprising:
discarding any of the median ratio values that are greater than 1.

7. The method of claim 1, further comprising:
obtaining, from the machine learning model, respective labels for frames of the displacement values, each label indicating an apnea event, a hypopnea event, or a no-event.

8. A device for apnea-hypopnea detection, comprising:
a processor configured to execute instructions to:
obtain accelerometer data from an accelerometer configured to measure micro-movements that are due to respiration;
obtain displacement values from the accelerometer data, wherein the displacement values correspond to peaks in the accelerometer data;
obtain features using the accelerometer data; and
obtain an apnea-hypopnea index (AHI) from a machine learning model that uses the features as inputs.

9. The device of claim 8, wherein to obtain the displacement values from the accelerometer data comprises to:
obtain respective displacement values in frames of the accelerometer data, wherein each frame corresponds to a predefined time window.

10. The device of claim 8, wherein to obtain the features comprises to:
obtain respective bin count data for bins,
wherein each bin corresponds to a respective consecutive number of value drops in the displacement values, and
wherein the processor obtains the respective bin count data for a bin by instructions to:
associate with the bin a count of the respective consecutive number of value drops in the displacement values.

11. The device of claim 8, wherein to obtain the features comprises to:
obtain displacement drop ratio values using the displacement values, wherein a displacement drop ratio value of a drop range [highest, . . . , lowest] identified in the displacement values and including n displacement values is obtained using a formula ((highest−lowest)/highest/n);
partition the displacement drop ratio values into groups, wherein each group includes a respective range of the displacement drop ratio values; and
use respective counts of the displacement drop ratio values in the groups as the features.

12. The device of claim 8, wherein to obtaining the features comprises to:
partition the displacement values into frames using a sliding window;
obtain median ratio values from the frames, wherein to obtain a median ratio value of a frame comprises to:
partition the frame into a first subframe that includes first displacement values and a second subframe that includes second displacement values; and
obtain the median ratio value as a ratio of a median value of the second displacement values divided by a median value of the first displacement values;
partitioning the median ratio values into groups, wherein each group includes a respective range of the median ratio values; and
use respective counts of the median ratio values in the groups as the features.

13. The device of claim 12, wherein the processor is further configured to execute instructions to:
discard any of the median ratio values that are greater than 1.

14. The device of claim 8, wherein the machine learning model further outputs respective labels for frames of the displacement values, each label indicating an apnea event, a hypopnea event, or a no-event.

15. A non-transitory computer readable medium storing instructions operable to cause one or more processors to perform operations for apnea-hypopnea detection, the operations comprising:
obtaining accelerometer data from an accelerometer configured to measure micro-movements that are due to respiration;
obtaining displacement values from the accelerometer data, wherein the displacement values correspond to peaks in the accelerometer data;
obtaining features using the accelerometer data; and
obtaining, from a machine learning model that uses the features as inputs, respective labels for frames of the displacement values, each label indicating an apnea event, a hypopnea event, or a no-event.

16. The non-transitory computer readable medium of claim 15, wherein obtaining the displacement values from the accelerometer data comprises:
obtaining respective displacement values in frames of the accelerometer data, wherein each frame corresponds to a predefined time window.

17. The non-transitory computer readable medium of claim 15, wherein obtaining the features comprises:
obtaining respective bin count data for bins,
wherein each bin corresponds to a respective consecutive number of value drops in the displacement values, and
wherein the respective bin count data for a bin is obtained by:
associating with the bin a count of the respective consecutive number of value drops in the displacement values.

18. The non-transitory computer readable medium of claim 15, wherein obtaining the features comprises:
obtaining displacement drop ratio values using the displacement values, wherein a displacement drop ratio value of a drop range [highest, . . . , lowest] identified in the displacement values and including n displacement values is obtained using a formula ((highest−lowest)/highest/n);
partitioning the displacement drop ratio values into groups, wherein each group includes a respective range of the displacement drop ratio values; and
using respective counts of the displacement drop ratio values in the groups as the features.

19. The non-transitory computer readable medium of claim 15, wherein obtaining the features comprises:
partitioning the displacement values into frames using a sliding window;
obtaining median ratio values from the frames, wherein obtaining a median ratio value of a frame comprises:
partitioning the frame into a first subframe that includes first displacement values and a second subframe that includes second displacement values; and
obtaining the median ratio value as a ratio of a median value of the second displacement values divided by a median value of the first displacement values;
partitioning the median ratio values into groups, wherein each group includes a respective range of the median ratio values; and
using respective counts of the median ratio values in the groups as the features.

20. The non-transitory computer readable medium of claim 15, further comprising:
obtaining an apnea-hypopnea index (AHI) from the respective labels.
